(19) United States
(12) Patent Application Publication
(10) Pub. No.: US 2010/0267085 A1
(43) Pub. Date: Oct. 21, 2010

(54) NOVEL PSEUDOCHLOROCOCCUM SPECIES AND USES THEREFOR

(75) Inventors: Qiang Hu, Chandler, AZ (US); Danzxiang Han, Mesa, AZ (US); Milton Summerfeld, Chandler, AZ (US)

Correspondence Address: DAVIS WRIGHT TREMAINE LLP - San Francisco, 505 MONTGOMERY STREET, SUITE 800, SAN FRANCISCO, CA, 94111, US

(73) Assignee: The Arizona Board of Regents, a Body Corporate Acting on Behalf of Arizona State University, Tempe, AZ (US)

(21) Appl. No.: 12/667,220

(22) PCT Filed: Jun. 29, 2007
(86) PCT No.: PCT/US07/15199

§ 371 Date: Jun. 7, 2010

(51) Int. Cl. C12P 21/06; C12N 1/13; C12M 1/00; C12P 7/64; C02F 3/32

(52) U.S. Cl. 435/69.1; 435/257.2; 435/292.1; 435/134; 210/602; 435/266

(57) ABSTRACT

The present invention relates to algal species and compositions, methods for identifying algae that produce high lipid content and possess CO₂ tolerance, and methods for using such algae for lipid isolation, wastewater remediation, waste gas remediation, and/or biomass production.

### FIELD OF THE INVENTION

The invention relates to algae, algae selection methods, and methods for using algae to make various products.

### BACKGROUND OF THE INVENTION

Global warming due to increases in CO₂ and other greenhouse gases (methane, chlorofluorocarbons, etc.) in the atmosphere, and widespread water pollution with nutrients (such as nitrogen and phosphate) and other contaminants, are major environmental concerns. Although many conventional techniques and approaches are available for pollution prevention and control, these methods are usually very expensive with high energy consumption. Large quantities of sludge and/or liquid wastes generated from these systems are difficult to deal with and may also pose the risk of creating secondary contamination. Oil, natural gas, coal, and nuclear energy are the predominant sources of energy used today and they are not sustainable. As energy consumption increases, the natural reserves of these nonrenewable fossil fuels shrink drastically. For instance, at the current rate of consumption, currently identified oil reserves will last approximately 50 years or less. Production and consumption of fossil fuels are also the major causes of regional and global air and water pollution.

Engineered bacterial system may be designed that can breakdown and remove nutrients and other contaminants from waste streams, but can not effectively convert and recycle waste nutrients into renewable biomass. Many oil crops such as soy, rapeseeds, sunflower seeds, palm seeds are a source of feedstock for biodiesel, but these crops can not adequately perform wastestream treatment.

### SUMMARY OF THE INVENTION

In a first aspect, the present invention provides isolated Pseudochlorococcum sp. compositions, wherein the isolated Pseudochlorococcum sp. genome comprises one or more nucleic acid sequence selected from the group consisting of SEQ ID NO:1 (ITS—1622 bp), SEQ ID NO:2 (rbcL—1160 bp), SEQ ID NO:3 (ITS1—928-1082 of ITS), SEQ ID NO:4 (ITS2—1247-1487 of ITS), and SEQ ID NO:5 (ITS—827 bp), or complements thereof.

In a second aspect, the present invention provides a substantially pure culture, comprising:

(a) a growth medium; and

(b) the isolated Pseudochlorococcum sp. composition of the first aspect of the invention.

In a third aspect, the present invention provides an algal culture system, comprising:

(a) a photobioreactor; and

(b) the substantially pure culture of the second aspect of the invention.

In a fourth aspect, the present invention provides methods for lipid isolation, wastewater remediation, waste gas remediation, and/or biomass production, comprising culturing a Pseudochlorococcum sp., wherein the Pseudochlorococcum sp. genome comprises one or more nucleic acid sequence selected from the group consisting of SEQ ID NO:1 (ITS—1622 bp), SEQ ID NO:2 (rbcL—1160 bp), SEQ ID NO:3 (ITS1—928-1082 of ITS), SEQ ID NO:4 (ITS2—1247-1487 of ITS), and SEQ ID NO:5 (ITS—827 bp), or complements thereof, wherein the culturing is carried out under conditions suitable for fatty acid isolation, wastewater remediation, waste gas remediation, and/or biomass production.

### BRIEF DESCRIPTION OF THE FIGURES

FIG. 1 Typical GC chart of fatty acid profile of Pseudochlorococcum sp.

FIG. 2 Effect of carbon dioxide on growth of Pseudochlorococcum sp. aerated with air containing either 1% or 15% CO₂. Cultures were maintained at 25±1° C. and light intensity of 175 μmol m⁻² s⁻¹. Cultures were grown in 300 ml capacity glass columns 68 cm long with an inner diameter of 2.3 cm.

FIG. 3 Effect of carbon dioxide on biomass yield of Pseudochlorococcum sp. (Culture conditions were the same as described for FIG. 1).

FIG. 4 Effects of carbon dioxide on the lipid content (a) and lipid yield (b) of Pseudochlorococcum sp (Culture conditions same as for FIG. 1).

FIG. 5 Effect of dairy wastewater (DWW) on growth of Pseudochlorococcum sp. grown in 300 ml capacity glass columns (68 cm long with an inner diameter of 2.3 cm) at 25±1° C., 1% CO₂, and continuous illumination of 170 μmol m⁻² s⁻¹.

FIG. 6 Effect of dairy wastewater on biomass yield of Pseudochlorococcum sp. grown in a glass column bioreactor (Growth conditions were the same as for FIG. 4).

FIG. 7 Effect of dairy wastewater on lipid content of Pseudochlorococcum sp. grown in a glass column bioreactor (Growth conditions were the same as for FIG. 4).

FIG. 8 Effect of dairy wastewater on lipid production by Pseudochlorococcum sp. grown in a glass column bioreactor (Growth conditions were the same as for FIG. 4).

FIG. 9 Growth kinetics of Pseudochlorococcum strain grown outdoors in flat panel bioreactors varying in light path. Culture conditions: maximum daily culture temperature was maintained at 29±12° C. by evaporative cooling. pH was 7.0 ˜8.0. Mixing and additional CO₂ supply were provided by compressed air stream enriched with 1% CO₂ through a perforated tube running through the bottom of the reactor.

FIG. 10 Lipid content of Pseudochlorococcum cells grown outdoors in the flat panel bioreactors of various light paths. (Culture conditions described in FIG. 8)

FIG. 11 Areal (a) and volumetric (b) production of Pseudochlorococcum biomass outdoors in the flat panel bioreactors of various light paths. (Culture conditions described in FIG. 8)

FIG. 12. Areal lipid yield and volumetric lipid yield of Pseudochlorococcum sp. grown in the different light-paths of the flat-panel photobioreactors outdoors. (Culture conditions described in FIG. 8)

FIG. 13 PCR products amplified from Pseudochlorococcum sp. A: DNA Ladder; B: ITS; and C: rbcL.

FIG. 14 Neighbor-joining (NJ) tree based on aligned nucleotide sequences for 827 bases in the regions of ITS from 22 OTUs belonging to Chlorophyta. The numbers above branches indicate the bootstrap values resolved in the majority-rule consensus tree of a bootstrap analysis based on 1000 replications. The non-significant values below 50 were not shown.

FIG. 15 Neighbor-joining (NJ) tree based on aligned nucleotide sequences for 1129 base pairs in the regions of rbcL from 20 OTUs belonging to Chlorophyta. The numbers above branches indicate the bootstrap values resolved in the majority-rule consensus tree of a bootstrap analysis based on 1000 replications. The non-significant values below 50 were not shown.

FIG. 16 Sequence alignment of 827 by region of ITS-rDNA segment for Pseudochlorococcum sp. and its phylogenetically closest-related species Desmodesmus multivariabilis var. turskensis Mary 8/18 T-1W (GeneBank Accession Number: DQ417). ITS1 (171-325) and ITS2 (488-729) are marked separately.

FIG. 17 Sequence Alignment of 1160 by of rbcL for Pseudochlorococcum sp. (PSP) and its phylogenetically closest-related species Neochloris sp. LCR (GeneBank Accession Number: EF012704).

### DETAILED DESCRIPTION OF THE INVENTION

In a first aspect, the present invention provides an isolated Pseudochlorococcum sp. composition, wherein the isolated Pseudochlorococcum sp. genome comprises one or more nucleic acid sequence selected from the group consisting of SEQ ID NO:1 (ITS—1622 bp), SEQ ID NO:2 (rbcL—1160 bp), SEQ ID NO:3 (ITS1—928-1082 of ITS), SEQ ID NO:4 (ITS2—1247-1487 of ITS), and SEQ ID NO:5 (ITS—827 bp), or complements thereof. As discussed in more detail below, each of these nucleic acid sequences serves as a marker for the novel Pseudochlorococcum sp. of the present invention, and distinguishes it from other Pseudochlorococcum strains.

The isolated Pseudochlorococcum sp. is useful for a variety of purposes, including but not limited to oil production, wastewater remediation, waste gas remediation, and production of other value-added biomass which can be used, for example, as animal feed and organic fertilizer. These uses are described in more detail below.

The alga of this first aspect of the invention was derived by a selection process from culture obtained from a water environment in the Phoenix metropolitan area.

Thus, the Pseudochlorococcum sp. derived may be naturally occurring, but previously not isolated, or may be derived by mutation caused by selective pressure during the selection process. As used herein, the Pseudochlorococcum sp. includes any strain with the identifying characteristic's recited.

As used herein the term “isolated” means that at least 90% of the algae present in the composition are of the recited Pseudochlorococcum genotype; in further embodiments, at least 95%, 98%, or 99% of the algae present are of the recited Pseudochlorococcum genotype. The isolated Pseudochlorococcum sp. can be cultured or stored in solution, frozen, dried, or on solid agar plates.

The Pseudochlorococcum sp. of this first aspect of the invention is characterized by (i) significant ammonia uptake, (ii) an ability to assimilate large quantities of nutrients selected from the group consisting of nitrogen, phosphorous, and inorganic carbon, and (iii) an ability to accumulate large quantities of biomass (including, but not limited to crude proteins, total lipids, total polysaccharides, and/or carotenoids (useful, for example, as livestock or aquaculture feed additive), or combinations thereof.

As used herein, the phrase “ability to grow” means that the algae capable of reproduction adequate for use in the methods of the invention under the recited conditions. As used herein, the phrase “an ability to assimilate large quantities of nutrients” means the following: for nitrogen (nitrate or ammonia/ammonium) removal from contaminated water and wastewater, at least 2 mg per liter of nitrogen as nitrate or ammonia per hour of treatment is regarded as a high removal rate (ie: assimilating large quantities of nutrients). In the case of CO₂ removal from power plant flue gas emissions of at least 2 grams of CO₂ per liter of algal culture per hour of cultivation time is regarded as a high removal rate.

In a second aspect, the present invention provides a substantially pure culture, comprising a growth medium; and isolated algae of the first aspect of the invention. As used herein, the term “growth medium” refers to any suitable medium for cultivating algae of the present invention. The algae of the invention can grow photosynthetically on CO₂ and sunlight, plus a minimum amount of trace nutrients. The volume of growth medium can be any volume suitable for cultivation of the algae for any purpose, whether for standard laboratory cultivation, to large scale cultivation for use in, for example, bioremediation, lipid production, and/or algal biomass 2.5 production. Suitable algal growth medium can be any such medium, including but not limited to BG-11 growth medium (see, for example, Rippka, 1979); culturing temperatures of between 10° and 38° C. are used; in other embodiments, temperature ranges between 15° and 30° are used. Similarly, light intensity between 20 μmol m⁻²s⁻¹ to 1000 μmol m⁻²s⁻¹ is used; in various embodiments, the range may be 100 μmol m⁻²s⁻¹ to 500 μmol m⁻²s⁻¹ or 150 μmol m⁻²s⁻¹ to 250 μmol m⁻²s⁻¹. Further, aeration is carried out with between 0% and 20% CO₂; in various embodiments, aeration is carried out with between 0.5% and 10% CO₂, 0.5% to 5% CO₂, or 0.5% and 2% CO₂.

For maintenance and storage purposes, Pseudochlorococcum sp. isolates are usually maintained in standard artificial growth medium. For regular maintenance purposes, the Pseudochlorococcum sp. isolates can be kept in liquid cultures or solid agar plates under either continuous illumination or a light/dark cycle of moderate ranges of light intensities (10 to 40 μmol m⁻² s⁻¹) and temperatures (18° C. to 25° C.).

The culture pH may vary from pH 6.5 to pH 9.5. No CO₂ enrichment is required for maintenance of Pseudochlorococcum sp. isolates. In various non-limiting examples, the temperature of culture medium in growth tanks is preferably maintained at from about 10° C. to about 38° C., in further embodiments, between about 20° C. to about 30° C.

In various embodiments, the growth medium useful for culturing Pseudochlorococcum sp. of the present invention comprises wastewater or waste gases. This growth medium is particularly useful when the Pseudochlorococcum sp. is used in a waste remediation process, although use of this growth medium is not limited to waste remediation processes. In one embodiment when wastewater is used to prepare the medium, it is from nutrient-contaminated water or wastewater (e.g., industrial wastewater, agricultural wastewater domestic wastewater, contaminated groundwater and surface water), or waste gases emitted from power generators burning natural gas or biogas, or flue gas emissions from fossil fuel fired power plants. In this embodiment, the Pseudochlorococcum sp. can be first cultivated in a primary growth medium, followed by addition of wastewater and/or waste gas. Alternatively, the Pseudochlorococcum sp. can be cultivated solely in the wastestream source. When a particular nutrient or element is added into the culture medium, it will be taken up and assimilated by the Pseudochlorococcum sp., just like other nutrients. In the end, both wastewater-containing and spiked nutrients are removed and converted into macromolecules (such as lipids, proteins, or carbohydrates) stored in Pseudochlorococcum sp. biomass. Typically, the wastewater is added to the culture medium at a desired rate. This water, being supplied from the waste water source, contains additional nutrients, such as phosphates, and/or trace elements (such as iron, zinc), which supplement growth of the Pseudochlorococcum sp. In one embodiment, if the wastewater being treated contains sufficient nutrients to sustain the Pseudochlorococcum sp. growth, it may be possible to use less of the growth medium. As the wastewater becomes cleaner due to Pseudochlorococcum sp. treatment, the amount of growth medium can be increased.

The major factors affecting wastewater feeding rate include: 1) Pseudochlorococcum sp. growth rate, 2) light intensity, 4) culture temperature, 5) initial nutrient concentrations in wastewater; 5) the specific uptake rate of certain nutrient/s; 6) design and performance of a specific bioreactor and 7) specific maintenance protocols.

In a third aspect, the present invention provides an algal culture system, comprising:

(a) a photobioreactor; and

(b) the substantially pure culture of the second aspect of the invention.

As used herein, a “photobioreactor” is a lab-scale or industrial-scale culture vessel in which algae grow and proliferate. For use in this aspect of the invention, any type of photobioreactor can be used, including but not limited to open raceways (i.e. shallow ponds (water level ca. 15 to 30 cm high) each covering an area of 10 to 5000 m² or larger, constructed as a loop in which the culture is circulated by a paddle-wheel (Richmond, 1986), closed systems, i.e. photobioreactors made of transparent tubes or containers in which the culture is mixed by either a pump or air bubbling (Lee 1986; Chaumont 1993; Richmond 1990; Tredici 2004), tubular photobioreactors 2.0 (for example, see Tamiya et al. (1953), Pirt et al. (1983), Gudin and Chaumont 1983, Chaumont et al. 1988; Richmond et al. 1993) and flat plate-type photobioreactors, such as those described in Samson and Leduy (1985), Ramos de Ortega and Roux (1986), Tredici et al. (1991, 1997) and Hu et al. (1996, 1998a,b). In this third aspect, the present invention provides systems of various designs, which can be used, for example, in methods for nutrient removal (described below) using the Pseudochlorococcum sp. of the invention.

The distance between the sides of a closed photobioreactor is the “light path,” which affects sustainable algal concentration, photosynthetic efficiency, and biomass productivity. In various embodiments, the light path of a closed photobioreactor can be between approximately 5 millimeters and 40 centimeters; between 50 millimeters and 30 centimeters, between 100 millimeters and 30 centimeters, between 1 centimeter and 30 centimeters, between 2 centimeters and 30 centimeters; between 2 centimeters and 20 centimeters, or between 2 centimeters and 10 centimeters. The most optimal light path for a given application will depend, at least in part, on factors including the specific algal strains to be grown and/or specific desired product/s to be produced.

In a fourth aspect, the present invention provides methods for lipid isolation, wastewater remediation, waste gas remediation, and/or biomass production, comprising culturing the Pseudochlorococcum sp. of the present invention, wherein the Pseudochlorococcum sp. genome comprises one or more nucleic acid sequence selected from the group consisting of SEQ ID NO:1 (ITS—1622 bp), SEQ ID NO:2 (rbcL—1160 bp), SEQ ID NO:3 (ITS1—928-1082 of ITS), SEQ ID NO:4 (ITS2—1247-1487 of ITS), and SEQ ID NO:5 (ITS—827 bp) or complements thereof, under conditions suitable to promote algal proliferation, and isolating lipids, removing nutrients from wastewater or waste gas, and/or extracting algal biomass. The methods can be carried out alone, or carried out in any combination. In one embodiment, methods for lipid isolation are carried out, where the lipid isolated can be a single lipid type, including, but not limited to, isolation of fatty acids, pigments (chlorophyll, carotenoids, etc.), sterols, vitamins A and D, or hydrocarbons, or combination thereof (such as total lipid). In a further embodiment, the methods comprise culturing the Pseudochlorococcum sp. of the present invention under conditions suitable for production of total lipid content of at least 20% of dry algal cell weight; in various embodiments, the total lipid content is at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more of the dry algal cell weight. As used herein, the “dry cell weight” is the total weight of the algal culture after concentrating and drying the algae from the culture. As discussed above, the methods of the first aspect of the invention can be used to select for algal isolates that produce a total lipid content of at least 40% of dry algal cell weight. Thus, those of skill in the art will be able to use such novel algae for lipid isolation, using any lipid extraction technique known in the art, including but not limited to the methods described below. Lipids, isolated via this method can be used for any purpose, including but not limited to biofuel production (including but not limited to biodiesel), detergent, biopolymers, and bioplastic.

In another embodiment, the methods comprise removing nutrients from a wastestream, comprising culturing the algal strain in a culture medium comprising at least 5% wastestream water, under conditions whereby nutrients in the wastestream are removed by the Pseudochlorococcum sp. of the present invention. In further embodiments, the culture medium comprises 10%, 20%, 30%, 40%, 50%, 60%, 70%, 80%, 90%, or 100% wastewater. Through this process up to 95% or more of the nutrients can be removed from the wastewater, resulting in nutrient levels below maximum contaminant levels set for individual contaminants by the U.S. Environmental Protection Agency (EPA). One non-limiting example of such wastewater is groundwater that may contain tens or hundreds of milligrams per liter of nitrogen as nitrate. The amounts of nitrate can be removed to below 10 mg nitrate-N L⁻¹ within one or several days, depending on initial nitrate concentration in the groundwater. The amounts of groundwater that can be purified by the methods of the invention depend on the initial concentrations of nutrients to be removed and the size of photobioreactor system used. In some cases, the groundwater may be spiked with trace amounts of phosphate (in a range of micro- or milligrams per liter) or microelements (such as Zn, Fe, Mn, Mg) in order to enable the algae to completely remove nitrate from the groundwater.

In another non-limiting embodiment, wastewater comes from Concentrated Animal Feeding Operations (CAFOs), such as dairy farms, which may contain high concentrations of ammonia (hundreds to thousands of milligrams per liter of nitrogen as ammonia) and phosphate (tens to hundreds of milligrams per liter of phosphorous as phosphate). Full-strength CAFO wastewater can be used as a “balanced growth medium” for sustaining rapid growth of selected algal strains in photobioreactors as described above. In some cases the CAFO wastewater can be diluted to a certain extent to accelerate growth and proliferation of the Pseudochlorococcum sp. of the present invention. As a result, ammonia and phosphate concentrations can be removed within one or several days, depending on initial concentrations of these nutrients. In contrast to the groundwater situation, no chemicals are required to be introduced into CAFO wastewater in order to reduce or eliminate ammonia and phosphate levels to meet the U.S. EPA standards. In another embodiment, wastewater is agricultural runoff water that may contain high concentrations (in a range of several to tens of milligrams per liter) of nitrogen in forms of nitrate and ammonia and phosphates. The Pseudochlorococcum sp. of the present invention can remove these nutrients to below the U.S. EPA's standards within one day or two, depending on initial concentrations of these nutrients and/or weather conditions. In case the nitrogen to phosphorous ratio is distant from the ratio of 15:1, addition of one chemical (either nitrates or phosphates) to balance the ratio is necessary to remove these nutrients from the wastewater.

In another embodiment of this fourth aspect, the methods comprise removing nutrients from a waste gas, comprising culturing the Pseudochlorococcum sp. of the present invention in a culture medium comprising waste gas, under conditions whereby nutrients in the waste gas are removed. In one embodiment, flue gas emissions provide a carbon source (in a form of carbon dioxide, or CO₂) for algal photosynthesis and waste nutrient removal. Flue gases may be those from any source, including but not limited to fossil fuel-burning power plants. Through the photosynthetic machinery, the Pseudochlorococcum sp. of the present invention cells fix CO₂ and convert it into organic macromolecules (such as carbohydrates, lipids, and proteins) stored in the cell. As a result, molecular CO₂ entering into the culture system disclosed above is removed and converted into algal biomass, and thus the gas released from the photobioreactor is significantly reduced in CO₂ (at least a 50% reduction).

In one embodiment, flue gases are delivered into a photobioreactor as disclosed above. One method involves injection of the flue gas directly into the photobioreactor at a flow rate that will sustain (0.1 to 0.5 liter of flue gas per liter of culture volume per minute) vigorous photosynthetic CO₂ fixation while exerting minimum negative effects due to lowering culture pH by dissolved NOₓ and SOₓ and/or certain toxic molecules such as the heavy metal mercury. Alternatively, the flue gas may be blended with compressed air at a certain ratio (flue gas to compressed air ratio may range from 0.1˜0.6 volume to 1 volume) and delivered into the photobioreactor through an aeration system. In a further embodiment, a liquid- or gas-scrubber system may be introduced to reduce or eliminate contaminant transfer from the gas-phase and accumulation of toxic compounds in the algal growth medium. In a further preferred embodiment, flue gases coming out from the power generator may be pre-treated with proton-absorbing chemicals such as NaOH to maintain an essentially neutral pH and turn potentially harmful NOₓ and SOₓ compounds into useful sulfur and nitrogen sources for algal growth. For example, a commercially available gas-scrubber can be incorporated into the photobioreactor system to provide algae with pretreated flue gas. In case of liquid wastes, pre-treatment includes but is not limited to 1) treat wastewater first through an anaerobic digestion process or natural or constructed wetland to remove most of the organic matter; 2) dilute wastewater 10% to 90% with regular ground or surface water, depending on concentrations of potential toxic compounds; 3) add certain nutrients (such as phosphorous and/or trace elements) to balance the nutrient composition for maximum sustainable nutrient removal and/or biomass production.

In a further embodiment of this fourth aspect of the invention, methods for producing biomass are provided, comprising culturing the Pseudochlorococcum sp. of the present invention and harvesting algal biomass components from the cultured algae. Such biomass can include, but is not limited to, crude proteins, total lipids (such as fatty acids), total polysaccharides, and/or carotenoids selected from the group consisting of lutein and beta-carotene (useful, for example, as livestock or aquaculture feed additive), or combinations thereof. In one embodiment, a multi-stage maintenance protocol is described to remove waste nutrients at the early stages, while inducing and accumulating high-value compounds (such as fatty acids, carotenoids) at later stages. In a further embodiment, algal biomass produced from the photobioreactor is used as feedstock for biodiesel production. In a further preferred embodiment, residues of algal mass after extraction of algal fatty acids will be used as animal feed or organic fertilizer additive. In another embodiment, carotenoid-rich algal biomass as a by-product of waste-stream treatment by algal strains grown in the photobioreactors described above is used as an animal-feed additive or a natural source of high-value carotenoids. Methods for algal biomass production and/or protein expression are well known in the art. See, for example: Hu, Q. (2004) Chapter 5: pp. 83-93. In Richmond A. (ed.) Handbook of Microalgal Culture, Blackwell Science Ltd, Oxford OX2 0EL, UK; Hu, Q. (2004) Chapter 12: Arthrospira (Spirulina) platensis, pp. 264-272. In Richmond A. (ed.) Handbook of Microalgal Culture, Blackwell Science Ltd, Oxford OX2 0EL, UK; Hu, Q., et al. (2000) Appl. Env. Microbiol. 66: 133-139; Hu, Q., et al. (1999) Acaryochloris marina. Biochim. Biophys. Acta, 1412: 250-261; Hu, Q., et al. (1998) Proc. Natl. Acad. Sci. USA, 95: 13319-13323; Hu, Q., et al. (1998) Acaryochloris marina. In: Garab G. (ed.) Photosynthesis: Mechanisms and Effects, Vol. I. 437-440, Kluwer Academic Publishers, Dordrecht, The Netherlands; Hu, Q., et al. (1998) J. Ferment. Biotechnol 85: 230-236; Hu, Q., et al. (1998) Eur. J. Phycol. 33: 165-171; Hu, Q., et al. (1998) Appl. Microbiol. Biotechnol. 49: 655-662; Iwasaki, I., et al. (1988) J. Photochem. Photobiol. B: Biology 44: 184-190; Hu, Q., et al. (1997) Eur. J. Phycol. 32: 81-86; Richmond, A. and Hu, Q. (1997) Appl. Biochem. Biotechnol. 63-65: 649-658; Hu, Q., et al. (1996) Biotechnol. Bioeng. 51: 51-60; Hu, Q., et al. (1996) J. Phycol. 32: 1066-1073; Hu, Q. and Richmond, A. (1996) J. Appl. Phycol. 8: 139-145; Gitelson, A., et al. (1996) Appl. Env. Microbiol. 62: 1570-1573; Hu, Q. and Richmond, A. (1995) In: Mathis P. (ed.) Photosynthesis: from Light to Biosphere, Vol. IV, 1037-1040, Kluwer Academic Publishers, The Netherlands; and Hu, Q. and Richmond, A. (1994) J. Appl. Phycol. 6: 391-396.

The present invention addresses environmental pollution control while producing renewable energy through novel algal reagents and methods. The Pseudochlorococcum sp. of the present invention can be used to produce biofuel (such as biodiesel) and/or rapidly remove nutrients from wastewater and/or waste gases (including but not limited to wastewater and power plant flue gases) and convert them into value-added compounds stored into algal biomass. The biomass can then be used, for example, as feedstock for production of liquid biofuel and/or fine chemicals, and used as animal feed, or organic fertilizer. The major advantages of the reagents and methods of the present invention over conventional bacteria-based systems are that it they only remove nutrients from wastewater or waste gas, but also recycle them in form of renewable biomass and fine chemicals, whereas bacterial systems strip off potentially valuable nitrate and/or ammonia into the atmosphere through nitrification and de-nitrification processes. Bacterial systems also usually generate large amounts of sludge which require proper disposal. Compared to natural and constructed wetland systems, the algae-based reagents and methods of the present invention are more efficient in terms of nutrient removal and biomass production. From the energy production side, the reagents and methods of the present invention are more efficient than conventional lipid crop production, producing up to 20 to 40 times more feedstock per unit area of land per year. The reagents and methods of the present invention can be applied in non-agricultural environments, such as arid and semi-arid environments (including deserts). Thus, the present technology will not compete with food/energy crop (or other) plants for limited agricultural land.

### Materials and Methods

### The Organism and Growth Conditions:

Starting algal cultures were obtained from a water environment in the Phoenix metropolitan area and maintained at 25° C. in BG-11 growth medium (Rippka, 1979).

### Optical Density and Dry Weight Measurements:

Algal cell population density was measured daily using a micro-plate spectrophotometer (SPECTRA max 340 PC) and reported as optical density at 660 nm wave length. The dry weight of algal mass was determined by filtration from 10-20 ml culture through a pre-weighed Whatman GF/C filter. The filter with algae was dried at 105° C. overnight and cooled to the room temperature in a desiccator and weighed.

### Chlorophyll Measurement:

A hot methanol extraction method was used (Azov (1982). The concentration was calculated using the Tailing coefficient:

Chlorophyll a(mg/L)=13.9(DO₆₆₅−DO₇₅₀)V/U

where DO665=optical density measured at 665 nm wavelength, DO750=optical density measured at 750 nm wavelength, V=total volume of methanol (ml), and U=volume of algal suspension (ml).

### Lipid Extraction:

The lipid extraction procedure was modified according to Bigogno, et al. (2002). Pseudochlorococcum cell biomass (100 mg freeze-dried) was added to a small glass vial sealed with Teflon screw cap and was extracted with methanol containing 10% DMSO, by warming to 40° C. for 1 h with magnetic stirring. The mixture was centrifuged at 3,500 rpm for five minutes. The resulting supernatant was removed to another clean vial and the pellet was re-extracted with a mixture of hexane and ether (1:1, v/v) for 30 minutes. The extraction procedure was repeated several times until negligible amounts of chlorophylls remained in the pellet. Diethyl ether, hexane and water were added to the combined supernatants, so as to form a ratio of 1:1:1:1 (v/v/v/v). The mixture was hand-shaken and then centrifuged at 3,500 rpm for 5 minutes. The upper phase was collected. The lower water phase was re-extracted twice with a mixture of diethyl ether:hexane (1:1, v/v). The organic phases were combined, and the solvents in the oil extract were completely removed by bubbling with nitrogen gas until the weight of the remaining oil extract was constant.

### Fatty Acid Analysis:

Fatty acids were analyzed by gas chromatography (GC) after direct transmethylation with sulphuric acid in methanol (Christie, 2003). The fatty acid methanol esters (FAMEs) were extracted with hexane containing 0.8% BHT and analyzed by a HP-6890 gas chromatography (Hewlett-Packard) equipped with HP7673 injector, a flame-ionization detector, and a HP-INNOWAX™ capillary column (HP 19091N-133, 30 m×0.25 mm×0.25 μm). Two (2) μL of the sample was injected in a split-less injection mode. The inlet and detector temperatures were kept at 250° C. and 270° C., respectively, and the oven temperature was programmed from 170° C. to 220° C. increasing at 1° C./min. High purity nitrogen gas was used as the carrier gas. FAMEs were identified by comparison of their retention times with those of the authentic standards (Sigma), and were quantified by comparing their peak areas with that of the internal standard (C17:0).

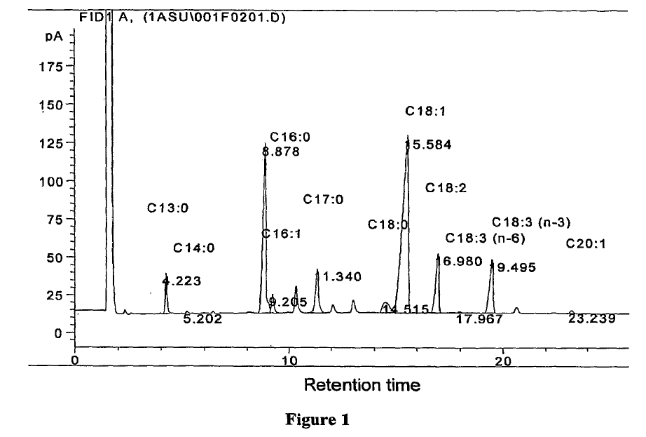

A typical GC chart of fatty acid profile of Pseudochlorococcum sp. is shown in FIG. 1. Each peak was marked as retention time and name of individual fatty acid. Some minor peaks between C16:1 and C18:0 (i.e., on both sides of the C17:0 peak) and between C18:3 (n−3) and C20:1 were not identifiable with the available standards and therefore were not labeled.

### Collection of Dairy Wastewater:

Dairy wastewater was collected at a dairy in Mesa, Ariz. (latitude N 33.35030, longitude W 111.65837) from a shallow wastewater pond consisting of piped dairy stall waste and overland runoff. A composite wastewater sample was collected from no fewer than three access points along the bank of a shallow wastewater pond. Wastewater was stored in a plastic container (5 gallons or larger) at 4° C.

Wastewater, in raw form, was brownish-red colored and contained undigested grains, grasses, soil and other unidentified solids. Before used for experiments, the dairy wastewater was filtered through a filtration system or centrifuged to remove particles and native species of algae at 5,000 rpm. The clear brown dairy wastewater was collected for assigned experiments. The wastewater was diluted to 5% wastewater (1:20 dairy wastewater to water), 25% wastewater (1:3 dairy wastewater to water), 50% wastewater (1:1 wastewater to water), 75% wastewater (3:1 wastewater to water), and 100% wastewater (undiluted wastewater) to meet various experimental needs.

### Experimental Design:

A 300-ml capacity glass column (68 cm long with an inner diameter of 2.3 cm) with a glass capillary rod placed down the center of the column to provide aeration was used to grow the alga. The top of the column was covered with a rubber stopper surrounded by loosely-fitting aluminum foil to prevent contamination among columns. Unless otherwise stated, a culture temperature of 25° C., a light intensity of 170 μmol m⁻² s⁻¹, and compressed air of 1% CO₂ were applied to glass columns throughout the experiment.

For experiments, log-phase cultures were harvested and centrifuged to remove the culture medium and re-suspended into small volume of sterilized distilled water for inoculation. Each treatment was run in triplicate. Deionized water was added daily to the column to compensate for water loss due to evaporation.

For nutrient removal experiments, 10 ml of culture suspension was collected from the column daily and centrifuged at 3,500 rpm for 10 minutes. The supernatant was pooled into small vial and frozen in a −20° C. freezer for nutrient analysis. The pellets were re-suspended into distilled water for dry weight measurement.

### High Carbon Dioxide Treatment:

For CO₂ treatment experiments, algal cells were grown in BG-11 growth medium either bubbled with air enriched with 1% CO₂, or air enriched with 15% CO₂.

### Outdoor Mass Culture Experiments:

To prepare a seed culture, 150 ml of stock culture of Pseudochlorococcum sp. was transferred from a flask to a 750 ml capacity glass column (68 cm long with an inner diameter of 5.7 cm), agitated with compressed air enriched with 1% CO₂. The seed culture was illuminated with a bank of daylight fluorescent lamps from one side of the column at a photon flux density of 100 μmol m⁻² s⁻¹ and at 25° C. When cell density of the culture reached 5×10⁷/ml, the culture was transferred to a flat-plate reactor measuring 210 cm×40 cm×13 cm, and containing 100 liters of BG-11 growth medium. The culture conditions for the flat-plate reactor were same as for the glass column reactors. When cell density of the flat-plate reactor reached 5×10⁶/ml, the 100 liters of culture was transferred to an outdoor thin panel photobioreactor.

The outdoor thin panel photobioreactor consisted of individual culture units varying in light path (i.e., culture depth), as desired. In this particular case, five different light-paths were used (2.5 cm, 5.0 cm, 10.0 cm, 20.0 cm and 30.0 cm). Given that all the individual reactors measured 210 cm long and 40 cm height, the total culture volume for the five different light-path reactors (e.g., 2.5 cm, 5.0 cm, 10.0 cm, 20.0 cm and 30.0 cm) was 21, 42, 84, 168 and 252 liters of culture, respectively.

Maximum daily culture temperature was maintained at 29±2° C. by evaporative cooling. Culture pH was maintained at 7.0˜8.0. Culture mixing and CO₂ supply were provided by compressed air enriched with 1% CO₂ through a perforated tube running through the bottom of the reactor.

### DNA Extraction, Amplification, and Sequencing:

Fifty (50) ml of cell culture was collected and centrifuged (3000 rpm×5 minutes) and then homogenized into powder in liquid nitrogen. Genomic DNA was extracted and purified with NucleoSpin Plant kit (MACHEREY-NAGEL Inc.). The ribosomal DNA internal transcribed spacer (ITS) (SEQ ID NO:1) and the large subunit of the Rubisco (rbcL) gene (SEQ ID NO:2) were used as the molecular markers for Pseudochlorococcum sp identification. PCR reactions contained 12.5 μl GoTaq Green Master Mix (ProMega), 200 ng template DNA and 0.5 μM primers (see Tablel) and H₂O in a final volume of 25 μl. PCR cycles for amplification of the region ITS were as follows: 1 cycle of 94° C., 5 min, 35 cycles of 94° C. 30 s, 50° C. 30 s, 72° C. 1 min 30 s and 1 cycle of 72° C. 10 min. PCR cycles for the amplification of rbcL were as follows: 1 cycle of 94° C., 5 min, 35 cycles of 94° C. 30 s, 55° C. 30 s, 72° C. 1 min 30 s and 1 cycle of 72° C. 10 min. PCR products are examined on 1.5% agarose. Two (2) μl of PCR products were cloned into the pCR®4-TOPO vector (Invitrogen). Plasmids for sequencing were extracted from the positive clones with the PureLink Quick Plasmid Miniprep kit (Invitrogen). The primers M13R and M13F were used for sequencing.

TABLE 1
Primers used for amplification of ITS and rbcL
from Pseudochlorococcum sp.

| Primers | Sequence (5′-3′) |
|---|---|
| ITS | |
| s15CH(F) | CTTAGTTGGTGGGTTGCC |
| | (SEQ ID NO: 9) |
| 15pl(R) | TTCRCTCGCCGTTACT |
| | (SEQ ID NO: 10) |
| rbcL | |
| RH1(F) | ATGTCACCACAAACAGAAACTAAAGC |
| | (SEQ ID NO: 11) |
| Cel 161R(R)² | CATGTGCAATACGTGAATACC |
| | 9SEQ ID NO: 12) |

### Phylogenetic Analysis Methods:

DNA sequences were aligned with Clustal W 1.83 and verified manually with Seaview. Phylogenetic trees were reconstructed with neighbor-joining (NJ) algorithm as implemented in Mega 3. The Kimura 2-parameter model was applied to calculate the substitution rate for reconstructing the phylogenetic trees.

### Results and Discussion:

### Isolation and Morphological Description of Pseudochlorococcum Sp.

The starting algal culture was collected from a public water pond in the Phoenix metropolitan area (Arizona) and isolated from the water sample by agar plating. Individual green colonies were then transferred into test tubes with screw-cap containing 10 ml BG-11 growth medium. Cultures were maintained at 20-25° C. with a light intensity of 20-40 μmol photons m⁻² s⁻¹. Cultures were examined weekly for growth by microscopy and spectrophotometry. Those mono-algal isolates that exhibited rapid growth and reproduction (any isolates that exhibited 1 to 3 doubling times per day under our culture conditions (e.g., BG-11 growth medium, 25° C., at light intensity of 170 μmol m⁻² s⁻¹, and aeration with 1˜2% CO₂)) were subjected to lipid content analysis. Only algal strains processing high lipid content (any isolates that possess a total lipid content of 40% or greater) were subjected to further screening for tolerance to high CO₂ concentrations and various wastewaters. One isolate that passed this screening process was identified as Pseudochlorococcum sp. based upon morphological features.

Pseudochlorococcum cells in a 2-week-old culture were ellipsoidal, with a single, thin parietal chloroplast (having the appearance of a thin, green rim) with at least 1 pyrenoid (additional pyrenoids may or may not occur with age). Cells were spherical in stationary phase cultures, and the chloroplast increased in size and filled the lumen, thereby causing old cells to resemble Chlorococcum. In a stationary phase, chloroplast was fissured but in young cells the chloroplast was always continuous. Large vacuoles were present, usually 1-2 in young cells, and additional vacuoles developed in older, spherical cells. Reproduction occurred only by 2 to 8 autospores, formed by successive bipartition(s). The genus Pseudochlorococcum was established by Archibald in 1970. At the beginning, only two species, Pseudochlorococcum typicum and Pseudochlorococcum polymorphum, were assigned to this genus. The major morphological features of this new isolate are similar to both Pseudochlorococcum typicum and Pseudochlorococcum polymorphum. However, the new isolate culture was dark green, whereas that of Pseudochlorococcum typicum and Pseudochlorococcum polymorphum were grass green during log- and stationary-phases. Under various stress conditions (such as high light, nutrients depletion or drought), cells of the new isolate tended to accumulate extraplastidic secondary carotenoids and high lipid content, making the culture orange to red color, whereas Pseudochlorococcum typicum and Pseudochlorococcum cells turned only a yellow color. Accordingly, this new isolate was assigned to be a new species of Pseudochlorococcum.

### Effect of Co₂ Concentration on Growth and Biomass Productivity

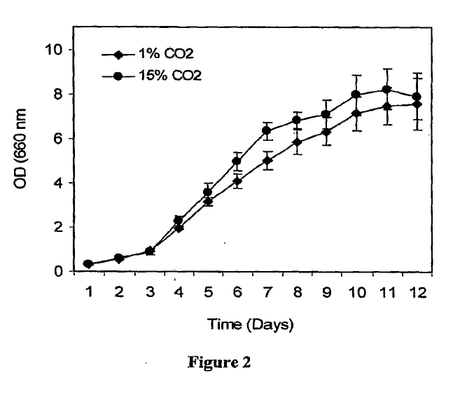

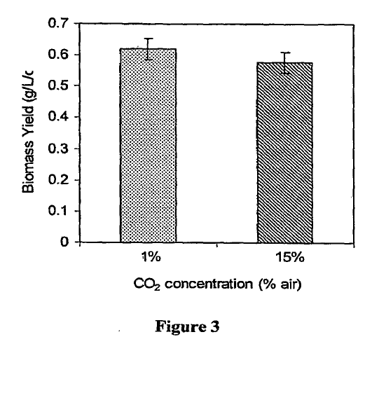

The Pseudochlorococcum strain can grow at a high CO₂ concentration (i.e., 15% CO₂) at a growth rate similar to that at 1% CO₂ commonly applied to algal cultures (FIG. 2). This CO₂ level is equivalent to that typically occurring in flue gases emitted from fossil fuel power plants. In a batch culture mode, the biomass productivity of the Pseudochlorococcum strain grown in a glass column reactor at 15% CO₂ was 570±50 mg l⁻¹ d⁻¹, similar to 610±70 mg l⁻¹ d⁻¹ obtained from cultures grown at 1% CO₂ (FIG. 3).

### Effect of CO₂ Concentration on Cellular Lipid Content and Lipid Productivity

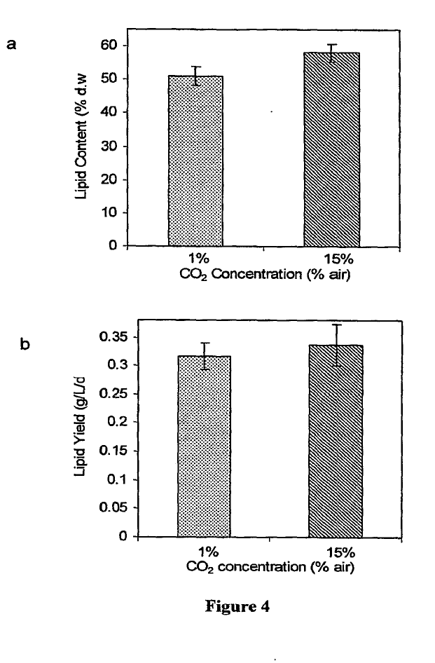

There was little effect of CO₂ concentrations on cellular lipid (fatty acid) content or lipid production of Pseudochlorococcum sp. As used herein, “content” refers to cellular lipid content at a point in time; lipid “production rate” or lipid “productivity” or “yield” refers to amount of lipid produced per unit culture volume or reactor illuminated surface area per time (day) of Pseudochlorococcum sp. When Pseudochlorococcum cultures were maintained in the glass column bioreactors supplied with 1% or 15% CO₂ under given culture conditions, the cellular oil content was 51˜56% of dry weight (FIG. 4a). Likewise, the volumetric productivity of oil was about 320±40 mg l⁻¹ d⁻¹ when Pseudochlorococcum cultures were provided with either level of CO₂ (FIG. 4b).

### Effect of Wastewater Concentration on Growth and Biomass Productivity

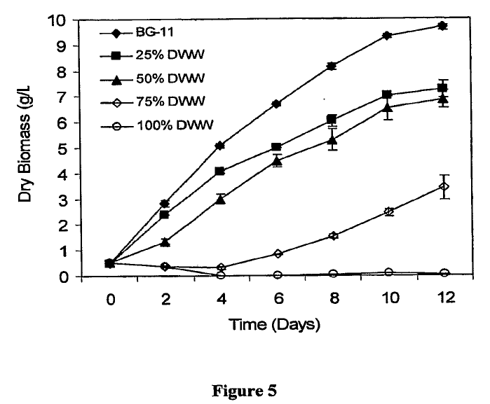

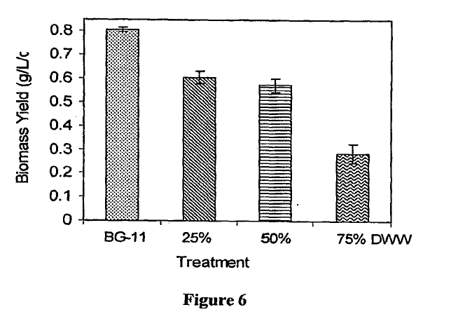

The Pseudochlorococcum strain has the ability to thrive in wastewater from various sources, such as nutrient-contaminated groundwater, agriculture runoff, and animal feeding operation wastewater. No additional nutrient chemicals were added to the culture, suggesting that the dairy wastewater contained nutrients necessary for sustaining algal growth and reproduction. FIG. 5 shows growth of Pseudochlorococcum sp. maintained in various concentrations of dairy wastewater (i.e., 25%, 50%, 75%, and 100% wastewater). While little growth occurred in cultures supplied with 100% dairy wastewater, Pseudochlorococcum cells did grow in 75% wastewater, albeit at much reduced growth rate. As the concentration of the wastewater decreased from 75% to 50% and further to 25% by dilution with tap water, growth was much improved (FIG. 5). As a result, a reverse relationship between wastewater concentration and biomass productivity of Pseudochlorococcum cells was observed: biomass productivity increased from 290±40 mg l⁻¹ d⁻¹ to about 800±60 mg l⁻¹ d⁴ as the wastewater concentration decreased from 100% to 25% (FIG. 6).

### Effect of Wastewater Concentration on Lipid Content and Lipid Productivity

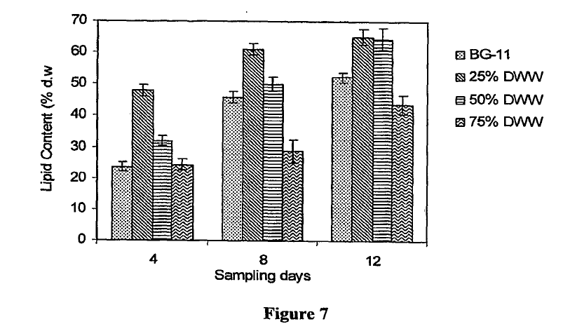

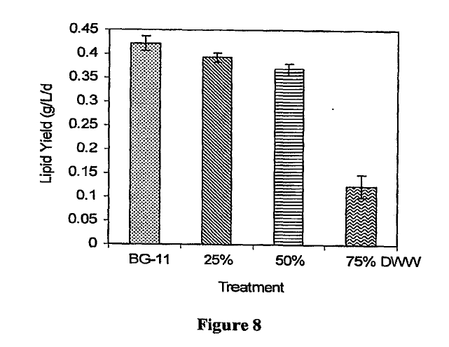

The concentration of dairy wastewater did affect the lipid content of algal biomass. The highest percentage of lipid was obtained in cultures maintained in 25% dairy wastewater. As the concentration of wastewater increased from 25% to 50% and to 75%, the cellular lipid content decreased (FIG. 7). As the cellular lipid content of the cells from 25% wastewater was significantly higher than that from BG-11 growth medium, it suggests that the wastewater may contain certain elements/compounds that stimulate biosynthesis and accumulation of lipid while at the same time somewhat inhibited growth. As a tradeoff, the lipid productivity was similar in cultures maintained in both BG-11 growth medium and 25% wastewater (FIG. 8).

### Fatty Acid Composition of Pseudochlorococcum Sp.

Table 2 shows the fatty acid composition of Pseudochlorococcum sp. grown in BG-11 growth medium. The major fatty acids (more than 95% of the total fatty acids in the cell) were C16 and C18.

TABLE 2
Fatty acid profile of Pseudochlorococcum sp.

| | Pseudochlorococcum sp. |
|---|---|
| Fatty acids | (% of total fatty acids) |
| C13:0 | 2.5 |
| C14:0 | 0.2 |
| C16:0 | 21.3 |
| C16:1 | 3.5 |
| C16:2 | 1.3 |
| Unidentified peak-1 | 3.3 |
| Unidentified peak-2 | 2.0 |
| C18:0 | 8.1 |
| C18:1 | 48.6 |
| C18:2n-6 | 5.7 |
| C18:3n-3 | 9.5 |
| C18:3n-6 | 0.1 |
| C18:4n-6 | 0.5 |
| C20:0 | 0.4 |
| C20:1 | 0.5 |
| TFA (% dry weight) | 52.4 |

### Outdoor Mass Culture of Pseudochlorococcum Sp.

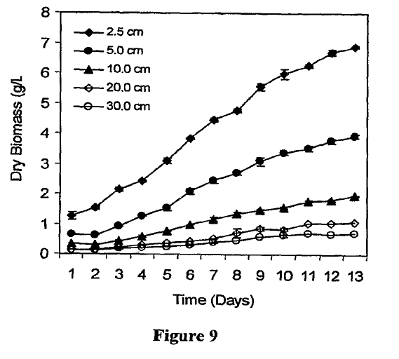

The Pseudochlorococcum strain was able to grow vigorously in photobioreactors of various designs (such as open raceway pond, vertical columns, and large flat panel reactors) to produce lipid-rich cell biomass under outdoor environmental conditions. The cultivation of the Pseudochlorococcum strain has been evaluated in a flat panel photobioreactor outdoors throughout the year. The results obtained indicate that Pseudochlorococcum sp. can thrive at a minimum culture temperature as low as 0° C. or even below 0° C. during the winter season, or in solar radiation as high as ca. 2,500 μmol m⁻² s⁻¹ at noon of a typical summer day in the Phoenix metropolitan area. FIG. 9 shows algal growth as a function of reactor light paths ranging from 2.5 cm to 30.0 cm. As indicated by the increase in algal dry biomass per culture volume over a 13-day period, the shorter the reactor light path, the greater the specific growth rate and thus the greater the maximum volumetric cell density reached at the end of cultivation.

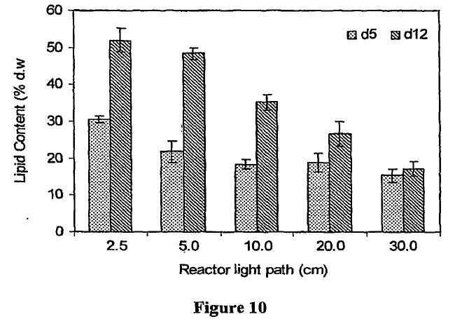

Reactor light path not only affected growth and final cell density of Pseudochlorococcum sp., but also affected cellular biochemical composition of the alga. FIG. 10 provides an example of total lipid content being largely affected by reactor light path. As reactor light path decreased from 30.0 cm to 2.5 cm, the total cellular lipid content increased from 18±2% to 51±6% of dry weight during a 13 day period of cultivation.

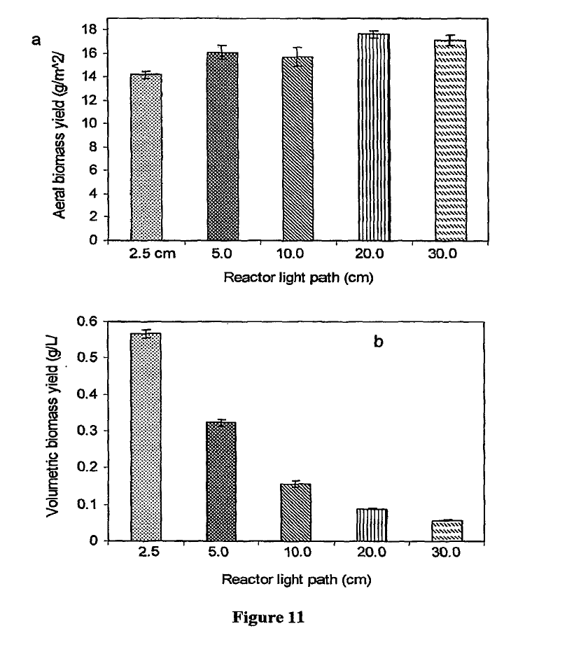

As a result, biomass productivity of Pseudochlorococcum or total lipid content was largely affected by reactor light path. On a per culture surface area basis, the difference in terms of productivity of Pseudochlorococcum biomass was minimum as the reactor light path decreased from 30.0 cm to 2.5 cm. However, on a volumetric basis, the shorter the reactor light path the greater the volumetric biomass yield (FIGS. 11a and b).

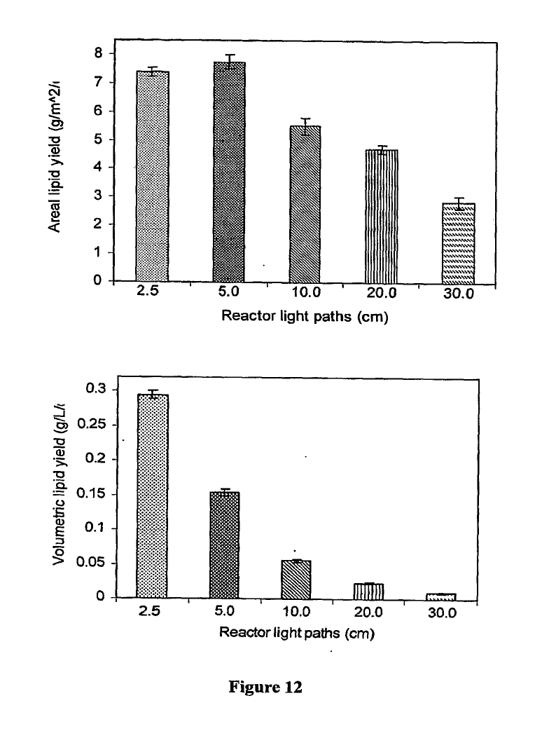

Photobioreactor light path exerted a more profound effect on productivity of total cellular lipids/oil of Pseudochlorococcum cultures. Both areal and volumetric productivities of total lipids were higher as a flat panel reactor decreased from a broader light path (e.g., 30.0 cm) to a narrower light path (e.g., 2.5 cm). Accordingly, over 100% increase in areal productivity of total lipids was obtained in cultures maintained in a 2.5 cm flat panel reactor compared to a 30.0 cm reactor, or more than 10 times higher total lipids productivity when calculated on a volumetric basis (FIG. 12 a and b).

In conclusion, the Pseudochlorococcum strain can grow and produce lipid as high as ca. 50% of dry weight with a production rate of more than 7 g m⁻² d⁻¹ in the flat panel photobioreactor tested for this disclosure under outdoor environmental conditions.

### DNA Markers for Identification of Pseudochlorococcum Sp.

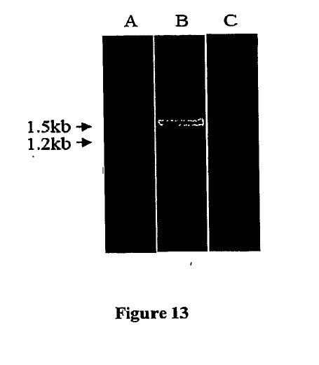

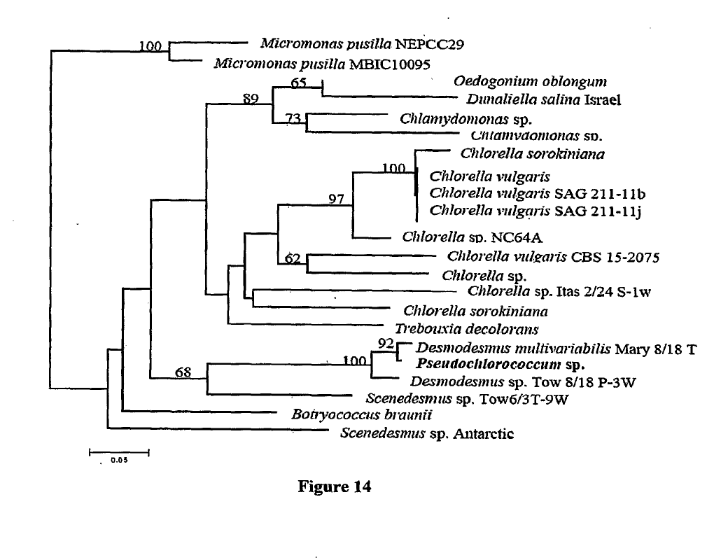

A 1623-bp ITS segment (SEQ ID NO:1) was amplified from Pseudochlorococcum sp., indicated by agarose gel electrophoresis (FIG. 13). The ITS segment consists of 3′ end of 18S rDNA (1-927) (SEQ ID NO:6) with an intron of 409 bp (491-899), ITS1 (928-1082) (SEQ ID NO:3), 5.8S rDNA (1083-1246) (SEQ ID NO:7), ITS2 (1247-1487) (SEQ ID NO:4) and 5′ end of 28S rDNA (1489-1622) (SEQ ID NO:8). No identical nucleotide sequence can be found by a BLAST searching in the National Center for Biotechnology Information (NCBI) databases. The phylogenetic relationships of 22 Chlorophyta taxa were inferred based on 827 base pairs of the ITS regions (SEQ ID NO:5). As shown in FIG. 14, Pseudochlorococcum sp. was clustered with Desmodesmus forming a clade supported by the Bootstrap analysis with high confidence. Based upon the morphological characteristics, the Pseudchlorococcum genus belongs to the class Chlorococcales, characterized with a single parietal chloroplast and being azoosporic. However, the ITS sequence analysis suggests that the Pseudchlorococcum strain may be phylogenetically related to Sphaeropleales species and supposedly originated from an ancestor shared with Desmodesmus. Whether all Pseudochlorococcum species fall into the same clade remains to be determined. It is also possible that the Pseudochlorococcum genus may be composed of a number of species that are genetically heterogenous.

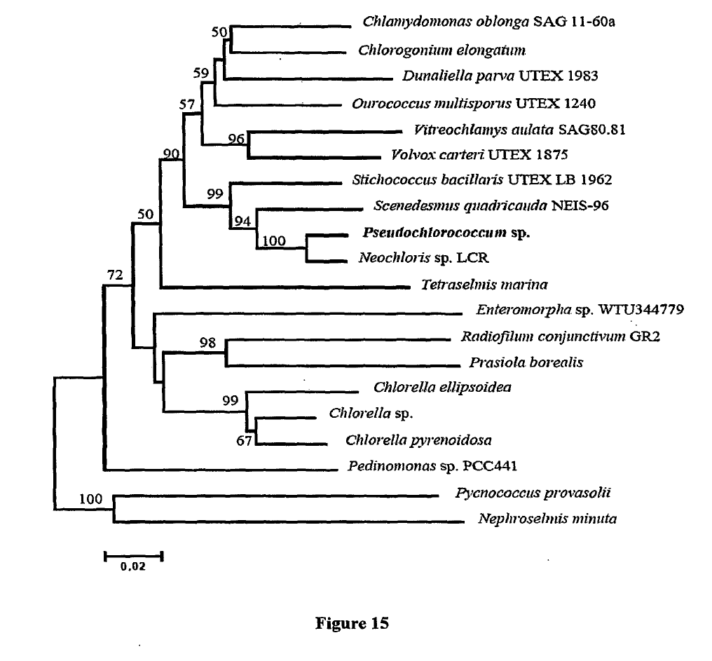

A 1160-bp rbcL segment (SEQ ID NO: 2) was also amplified from Pseudochlorococcum sp. and the sequence showed high identity with the strains belonging to the Sphaeropleales as indicated by a BLAST search in NCBI. Most mutations occurred at the third position of codons among closely-related strains. The phylogenetic tree reconstructed on 1160 base pairs of 20 different Chlorophyta taxa (FIG. 15) supports Pseudochlorococcum sp. being related to some Sphaeropleales species, which is congruent with the phylogenetic relationship based on the sequences of the ITS region.

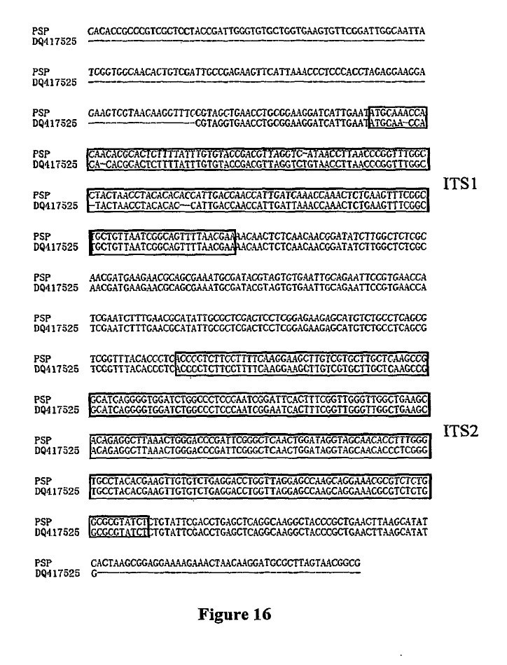

The internal transcribed spacer 1 (ITS1) is the non-coding segment located between 18S rDNA and 5.85 rDNA; the internal transcribed spacer 2 (ITS2) is located between 5.8S rDNA and 28S rDNA. The ITS1 of the newly-isolated Pseudochlorococcum sp. being 155 by in length shares 95% identity with the sequence of its closely-related Desmodesmus multivariabilis var. turskensis Mary 8/18 T-1W. As shown in FIG. 16 six indels (insertion and deletion) occurred in the ITS1 region. The identity of ITS2 sequence shared by these two species is about 99%, slightly more conserved than ITS1. Therefore, Pseudochlorococcum sp. is distinguishable to its closely-related species at the fast-evolving DNA region ITS1.

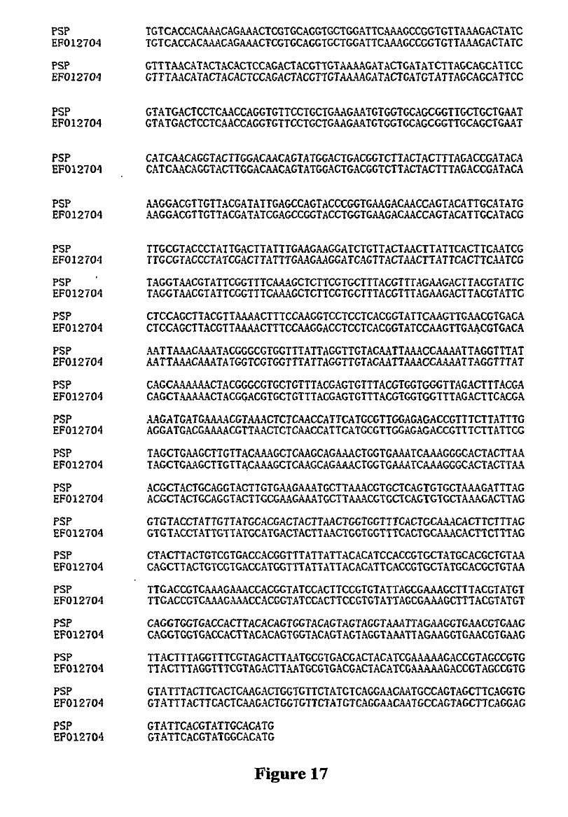

The rbcL sequence of Pseudochlorococcum sp. shows 97% identity with Neochloris sp. LCR (FIG. 17). Only two of the mutations (843A/G, 1153T/G) are non-synonymous and others that occurred at the third position of the codons are synonymous. Thus, the rbcL region can be used to distinguish Pseudochlorococcum sp. from its closely-related species.

### REFERENCES

Archibald, P. A. 1970. Pseudochlorococcum, A new chlorococcalean genus. J. Phycol. 6: 127-132.
Azov, Y. 1982. Effect of pH on organic carbon uptake in algal culture. Applied and Envir. Microbiol. 43(6): 1300-1306.
Bigogno, C., Khozin-Goldberg, I., Boussiba, S., Vonshak, A. and Cohen, Z. 2002. Lipid and fatty acid composition of the green oleaginous alga Parietochloris incise, the richest plant source of arachidonic acid. Phytochemistry. 60: 497-503.
Chaumont D. (1993) Biotechnology of algal biomass production: a review of systems for outdoor mass culture. J Appl Phycol 5: 593-604
Chaumont D., Thepenier C. and Gudin C. (1988) Scaling up a tubular photobioreactor for continuous culture of Porphyridium cruentum—from laboratory to pilot plant, pp. 199-208. In Stadler T, Morillon J, Verdus M S, Karamanos W, Morvan H, Christiaen D (eds.), Algal Biotechnology. Elsevier Applied Science, London.
Christie, W. W. 2003. Lipid analysis: Isolation, separation, identification, and structural analysis of lipids. Bridgwater, UK: Oily Press.
Cornwell, D. A. (1990). Air Stripping and Aeration, in Water Quality and Treatment, McGraw Hill Inc., New York, pg. 229-268.
Fernandez, F. G., Camacho, F. G., Perez, J. A., Sevilla, J. M., Grima, E. M. (1998). Modeling of biomass productivity in tubular photobioreactors for microalgal cultures: effects of diluation rate, tube diameter, and solar irradiance, Biotech. And Bioeng., 58:6:605-616.
Gitelson A., Hu Q. and Richmond A. (1996) Photic volume in photobioreactors supporting ultrahigh population densities of the photoautotroph Spirulina platensis. Applied and Environmental Microbiology 62: 1570-1573.
Gudin C. and Chaumont D. (1991) Cell fragility—the key problem of microalgae mass production in closed photobioreactors. Bioresource Technol. 38: 145-151.
Hu Q. and Richmond A. (1994) Optimizing the population density of Isochrysis galbana grown outdoors in a glass column photobioreactor. Journal of Applied Phycology 6: 391-396.
Hu Q., Faiman D. and Richmond A. (1998a) Optimal orientation of enclosed reactors for growing photoautotrophic microorganisms outdoors. Journal of Fermentation and Biotechnology 85: 230-236.
Hu Q., Guterman H. and Richmond A. (1996a) A flat inclined modular photobioreactor (FIMP) for outdoor mass cultivation of photoautotrophs. Biotechnology and Bioengineering 51: 51-60.
Hu Q., Guterman H. and Richmond A. (1996b) Physiological characteristics of Spirulina platensis cultured at ultrahigh cell densities. Journal of Phycology 32: 1066-1073.
Hu Q., Kurano N., Iwasaki I., Kawachi M. and Miyachi S. (1998b) Ultrahigh cell density culture of a marine green alga, Chlorococcum littorale in a flat plate photobioreactor. Applied Microbiology and Biotechnology 49: 655-662.
Hu Q., Yak Z. and Richmond A. (1998c) Combined effects of light intensity, light-path and culture density on output rate of Spirulina platensis (Cyanobacteria). European Journal of Phycology 33: 165-171.
Hu Q. 2004. Industrial production of microalgal cell-mass and secondary products—major industrial species: Arthrospira (Spirulina) platensis. Pp. 264-272. In: Richmond A. (ed.) Handbook of microalgal culture: biotechnology and applied Phycology, Blackwell Science Ltd., Oxford, UK.
Iwasaki I., Hu Q., Kurano N. and Miyachi S. (1988) Effect of extremely high-CO₂ stress on energy distribution between photosystem I and photosystem II in a ‘High-CO₂’ tolerant green alga, Chlorococcum littorale and the intolerant green Alga Stichococcus bacillaris. Journal of Photochemistry and Photobiology B: Biology 44/3: 184-190.
Lee Y. K. (1986) Enclosed bioreactor for the mass cultivation of photosynthetic microorganism: the future trend. Trends Biotechnol. 4: 186-189.
Lee Y. K., Ding S. Y., Low C. S, and Chang Y. C. (1995) Design and performance of an α-type tubular photobioreactor for mass cultivation of microalgae. J. Appl. Phycol. 7:47-51
Pirt S. J., Lee Y. K., Walach M. R., Pirt M. W., Balyuzi H. H. M. and Bazin M. J. (1983) A tubular bioreactor for photosynthetic production of biomass from carbon dioxide: design and performance. J. Chem. Tech. Biotechnol. 33: 35-58.
Ramos de Ortega and Roux J. C. (1986) Production of Chlorella biomass in different types of flat bioreactors in temperate zones. Biomass 10: 141-156.
Richmond A. (1990) Large scale microalgal culture and applications. In: Round/Chapman (eds) Progress in Phycological Research Biopress, London, 7: 1-62
Richmond A. (1992) Open systems for mass production of photoautotrophic microalgae outdoors: physiological principles. J. Appl. Phycol. 4: 281-286
Richmond A. and Hu Q. (1997) Principles for utilization of light for mass production of photoautotrophic microorganisms. Applied Biochemistry and Biotechnology. 63-65:649-658.
Richmond A., Boussiba S., Vonshak A. and Kopel R. (1993) A new tubular reactor for mass production of microalgae outdoors. J. Appl. Phycol. 5: 327-332.
Rippka R, Deruelles J, Waterbury J B, Herdman M, Stonier R Y (1979) Generic assignments, strain histories and properties of pure cultures of cyanobacteria. J Gen Microbiol 111:1-61.
Samson R & Leduy A (1985) Multistage continuous cultivation of blue-green alga Spirulina maxima in the flat tank photobioreactors with recycle. Can. J. Chem. Eng. 63: 105-112.,
Silva, H. J., Cortinas T. and Ertola R. J. 1987. Effect of hydrodynamic stress on Dunaliella growth. J. Chem. Tech. Biotechnol. 40: 253-264.
Tamiya H. (1957) Mass culture of algae. Ann Rev. Plant Physiol. 8: 309-334.
Torzillo G., Carlozzi P., Pusharaj B., Montani E. and Materassi R. (1994) A two-plane tubular photobioreactor for outdoor culture of Spirulina. Biotechnol. Bioeng. 42: 891-898.
Tredici M. R. and Materassi R. (1992) From open ponds to vertical alveolar panels: the Italian experience in the development of reactors for the mass cultivation of photoautotrophic microorganisms. J. Appl. Phycol. 4: 221-31.
Tredici M. R., Carlozzi P., Zittelli G. C. and Materassi R. (1991) A vertical alveolar panel (VAP) for outdoor mass cultivation of microalgae and Cyanobacteria. Bioresource Technol. 38: 153-159.
Tredici M. (2004) Mass production of microalgae: photobioreactors, pp. 178-214. In: Richmond A. (ed.) Handbook of microalgal culture: biotechnology and applied Phycology, Blackwell Science Ltd., Oxford, UK.
Watanabe Y., Joel de la Noue. And Hall D. O. (1995) Photosynthetic performance of a helical tubular photobioreactor incorporating the cyanobacterium Spirulina platensis. Biotechnol Bioeng 47: 261-269
Xiaoling M. and Qingyu, W. 2006. Biodiesel production from heterotrophic macroalgal oil. Bioresource Techn. 97: 841-846.
Xiu, Z-L, Zeng, A-P, Deckwer, W-D (1998). Multiplicity and stability analysis of microorganisms in continuous culture: effects of metabolic overflow and growth inhibition, Biotechnology and Bioeng., 57:3:251-261.

1. An isolated Pseudochlorococcum sp. composition, wherein the isolated Pseudochlorococcum sp. genome comprises one or more nucleic acid sequence selected from the group consisting of SEQ ID NO:1, SEQ ID NO:2, SEQ ID NO:3, SEQ ID NO:4, and SEQ ID NO:5, or complements thereof.

2. The isolated Pseudochlorococcum sp. composition of claim 1, wherein the isolated Pseudochlorococcum sp. genome comprises the nucleic acid sequence of SEQ ID NO:3.

3. The isolated Pseudochlorococcum sp. composition of claim 1, wherein the isolated Pseudochlorococcum sp. genome comprises the nucleic acid sequence of SEQ ID NO:4.

4. The isolated Pseudochlorococcum sp. composition of claim 1, wherein the isolated Pseudochlorococcum sp. genome comprises the nucleic acid sequence of SEQ ID NO:5.

5. The isolated Pseudochlorococcum sp. composition of claim 1, wherein the isolated Pseudochlorococcum sp. genome comprises the nucleic acid sequence of SEQ ID NO:1.

6. A substantially pure culture, comprising:
(a) a growth medium; and
(b) the isolated Pseudochlorococcum sp. composition of claim 1.

7. An algal culture system, comprising:
(a) a photobioreactor; and
(b) the substantially pure culture of claim 6.

8. A method for lipid isolation, comprising culturing a Pseudochlorococcum sp., wherein the Pseudochlorococcum sp. genome comprises one or more nucleic acid sequence selected from the group consisting of SEQ ID NO:1, SEQ ID NO:2, SEQ ID NO:3, SEQ ID NO:4, and SEQ ID NO:5, or complements thereof, wherein the culturing is done under conditions suitable for proliferation of the Pseudochlorococcum sp., and extracting lipid from the Pseudochlorococcum sp.

9. A method for removing nutrients from wastewater, comprising culturing a Pseudochlorococcum sp., wherein the Pseudochlorococcum sp. genome comprises one or more nucleic acid sequence selected from the group consisting of SEQ ID NO:1, SEQ ID NO:2, SEQ ID NO:3, SEQ ID NO:4, and SEQ ID NO:5, or complements thereof, wherein the culturing is done under conditions suitable for proliferation of the Pseudochlorococcum sp., and wherein the culturing is carried out in a culture medium comprising at least 5% wastewater, under conditions whereby nutrients in the wastewater are removed by the Pseudochlorococcum sp.

10. A method for removing nutrients from waste gas, comprising culturing a Pseudochlorococcum sp., wherein the Pseudochlorococcum sp. genome comprises one or more nucleic acid sequence selected from the group consisting of SEQ ID NO:1, SEQ ID NO:2, SEQ ID NO:3, SEQ ID NO:4, and SEQ ID NO:5, or complements thereof, wherein the culturing is done under conditions suitable for proliferation of the Pseudochlorococcum sp., and wherein the culturing is carried out in a culture medium comprising waste gas, under conditions whereby nutrients in the waste gas are removed by the Pseudochlorococcum sp.

11. The method of claim 8, further comprising harvesting algal protein and/or biomass components from the cultured Pseudochlorococcum sp.

12. A method for producing biomass, comprising culturing a Pseudochlorococcum sp., wherein the Pseudochlorococcum sp. genome comprises one or more nucleic acid sequence selected from the group consisting of SEQ ID NO:1, SEQ ID NO:2, SEQ ID NO:3, SEQ ID NO:4, and SEQ ID NO:5, or complements thereof, wherein the culturing is carried out under conditions suitable for proliferation of the Pseudochlorococcum sp., and harvesting algal protein and/or biomass components from the cultured Pseudochlorococcum sp.

13. The method of claim 12, wherein the Pseudochlorococcum sp. genome comprises the nucleic acid sequence of SEQ ID NO:3.

14. The method of claim 12, wherein the Pseudochlorococcum sp. genome comprises the nucleic acid sequence of SEQ ID NO:4.

15. The method of claim 12, wherein the Pseudochlorococcum sp. genome comprises the nucleic acid sequence of SEQ ID NO:5.

16. The method of claim 12, wherein the Pseudochlorococcum sp. genome comprises the nucleic acid sequence of SEQ ID NO:1.

17. The method of claim 9, further comprising harvesting algal protein and/or biomass components from the cultured Pseudochlorococcum sp.

18. The method of claim 10, further comprising harvesting algal protein and/or biomass components from the cultured Pseudochlorococcum sp.

19. The method of claim 8, wherein the Pseudochlorococcum sp. genome comprises the nucleic acid sequence of SEQ ID NO:3.

20. The method of claim 8, wherein the Pseudochlorococcum sp. genome comprises the nucleic acid sequence of SEQ ID NO:4.


SEQUENCE LISTING (12 sequences)

SEQ ID NO: 1 - DNA, 1622 bp, Pseudochlorococcum sp.
cttagttggtgggttgccttgtcaggttgattccggtaacgaacgagacctcagcctttaaatagtcacggtcgctttttgcggttggtttgacttcttagagggacagttggcgtttagtcaacgaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattatcggtggcaacactgtcgattgccgagaagttcattaaaccctcccacctagaggaaggagaagtcgtaacaaggtttccgtagccatgcggatgtcagtcttaaaaggactggcaagtgggaacgaatgtatattctttcctgcaagactgtcaaattgccggaacatcctgttaggccactggtaccgccctctctaggaaactaggttgtggcaccgtagggaaacttgcgggtatggtaaaaatccagtggatagggacaatcggcagccaagccctgagggtaagcaattacctacgggtgcagttcacagactaaatggcagtcggctggcctgacggctggcttaagatatagtcgggccctaccgagaggtagcctataagaggacggcacctgtgcttgagagcttataggaggttgggtaatacttattgccgtaaggcattaagggctgctgaacagtggcctcaaacctggagtaaccgaggtgaacctgcggaaggatcattgaatatgcaaaccacaacacgcactcttttatttgtgtaccgacgttaggtcataaccttaacccggtttggcctactaacctacacacaccattgaccaaccattgatcaaaccaaactctgaagtttcggctgctgttaatcggcagttttaacgaaaacaactctcaacaacggatatcttggctctcgcaacgatgaagaacgcagcgaaatgcgatacgtagtgtgaattgcagaattccgtgaaccatcgaatctttgaacgcatattgcgctcgactcctcggagaagagcatgtctgcctcagcgtcggtttacaccctcacccctcttccttttcaaggaagcttgtcgtgcttgctcaagccggcatcaggggtggatctggccctcccaatcggattcactttcggttgggttggctgaagcacagaggcttaaactgggacccgattcgggctcaactggataggtagcaacacctttgggtgcctacacgaagttgtgtctgaggacctggttaggagccaagcaggaaacgcgtctctggcgcgtatctctgtattcgacctgagctcaggcaaggctacccgctgaacttaagcatatcactaagcggaggaaaagaaactaacaaggatgcgcttagtaacggcgagcgaaaagggcgaattcgtttaaacctgcaggactagt

SEQ ID NO: 2 - DNA, 1160 bp, Pseudochlorococcum sp.
tgtcaccacaaacagaaactcgtgcaggtgctggattcaaagccggtgttaaagactatcgtttaacatactacactccagactacgttgtaaaagatactgatatcttagcagcattccgtatgactcctcaaccaggtgttcctgctgaagaatgtggtgcagcggttgctgctgaatcatcaacaggtacttggacaacagtatggactgacggtcttactactttagaccgatacaaaggacgttgttacgatattgagccagtacccggtgaagacaaccagtacattgcatatgttgcgtaccctattgacttatttgaagaaggatctgttactaacttattcacttcaatcgtaggtaacgtattcggtttcaaagctcttcgtgctttacgtttagaagacttacgtattcctccagcttacgttaaaactttccaaggtcctcctcacggtattcaagttgaacgtgacaaattaaacaaatacgggcgtggtttattaggttgtacaattaaaccaaaattaggtttatcagcaaaaaactacgggcgtgctgtttacgagtgtttacgtggtgggttagactttacgaaagatgatgaaaacgtaaactctcaaccattcatgcgttggagagaccgtttcttatttgtagctgaagcttgttacaaagctcaagcagaaactggtgaaatcaaagggcactacttaaacgctactgcaggtacttgtgaagaaatgcttaaacgtgctcagtgtgctaaagatttaggtgtacctattgttatgcacgactacttaactggtggtttcactgcaaacacttctttagctacttactgtcgtgaccacggtttattattacacatccaccgtgctatgcacgctgtaattgaccgtcaaagaaaccacggtatccacttccgtgtattagcgaaagctttacgtatgtcaggtggtgaccacttacacagtggtacagtagtaggtaaattagaaggtgaacgtgaagttactttaggtttcgtagacttaatgcgtgacgactacatcgaaaaagaccgtagccgtggtatttacttcactcaagactggtgttctatgtcaggaacaatgccagtagcttcaggtggtattcacgtattgcacatg

SEQ ID NO: 3 - DNA, 155 bp, Pseudochlorococcum sp.
atgcaaaccacaacacgcactcttttatttgtgtaccgacgttaggtcataaccttaacccggtttggcctactaacctacacacaccattgaccaaccattgatcaaaccaaactctgaagtttcggctgctgttaatcggcagttttaacgaa

SEQ ID NO: 4 - DNA, 241 bp, Pseudochlorococcum sp.
accctcacccctcttccttttcaaggaagcttgtcgtgcttgctcaagccggcatcaggggtggatctggccctcccaatcggattcactttcggttgggttggctgaagcacagaggcttaaactgggacccgattcgggctcaactggataggtagcaacacctttgggtgcctacacgaagttgtgtctgaggacctggttaggagccaagcaggaaacgcgtctctggcgcgtatct

SEQ ID NO: 5 - DNA, 827 bp, Pseudochlorococcum sp.
cacaccgcccgtcgctcctaccgattgggtgtgctggtgaagtgttcggattggcaattatcggtggcaacactgtcgattgccgagaagttcattaaaccctcccacctagaggaaggagaagtcgtaacaaggtttccgtagctgaacctgcggaaggatcattgaatatgcaaaccacaacacgcactcttttatttgtgtaccgacgttaggtcataaccttaacccggtttggcctactaacctacacacaccattgaccaaccattgatcaaaccaaactctgaagtttcggctgctgttaatcggcagttttaacgaaaacaactctcaacaacggatatcttggctctcgcaacgatgaagaacgcagcgaaatgcgatacgtagtgtgaattgcagaattccgtgaaccatcgaatctttgaacgcatattgcgctcgactcctcggagaagagcatgtctgcctcagcgtcggtttacaccctcacccctcttccttttcaaggaagcttgtcgtgcttgctcaagccggcatcaggggtggatctggccctcccaatcggattcactttcggttgggttggctgaagcacagaggcttaaactgggacccgattcgggctcaactggataggtagcaacacctttgggtgcctacacgaagttgtgtctgaggacctggttaggagccaagcaggaaacgcgtctctggcgcgtatctctgtattcgacctgagctcaggcaaggctacccgctgaacttaagcatatcactaagcggaggaaaagaaactaacaaggatgcgcttagtaacggcg

SEQ ID NO: 6 - DNA, 927 bp, Pseudochlorococcum sp.
cttagttggtgggttgccttgtcaggttgattccggtaacgaacgagacctcagcctttaaatagtcacggtcgctttttgcggttggtttgacttcttagagggacagttggcgtttagtcaacgaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattatcggtggcaacactgtcgattgccgagaagttcattaaaccctcccacctagaggaaggagaagtcgtaacaaggtttccgtagccatgcggatgtcagtcttaaaaggactggcaagtgggaacgaatgtatattctttcctgcaagactgtcaaattgccggaacatcctgttaggccactggtaccgccctctctaggaaactaggttgtggcaccgtagggaaacttgcgggtatggtaaaaatccagtggatagggacaatcggcagccaagccctgagggtaagcaattacctacgggtgcagttcacagactaaatggcagtcggctggcctgacggctggcttaagatatagtcgggccctaccgagaggtagcctataagaggacggcacctgtgcttgagagcttataggaggttgggtaatacttattgccgtaaggcattaagggctgctgaacagtggcctcaaacctggagtaaccgaggtgaacctgcggaaggatcattgaata

SEQ ID NO: 7 - DNA, 164 bp, Pseudochlorococcum sp.
acaactctcaacaacggatatcttggctctcgcaacgatgaagaacgcagcgaaatgcgatacgtagtgtgaattgcagaattccgtgaaccatcgaatctttgaacgcatattgcgctcgactcctcggagaagagcatgtctgcctcagcgtcggtttacac

SEQ ID NO: 8 - DNA, 134 bp, Pseudochlorococcum sp.
tattcgacctgagctcaggcaaggctacccgctgaacttaagcatatcactaagcggaggaaaagaaactaacaaggatgcgcttagtaacggcgagcgaaaagggcgaattcgtttaaacctgcaggactagt

SEQ ID NO: 9 - DNA, 18 bp, Artificial
  Synthetic
cttagttggtgggttgcc

SEQ ID NO: 10 - DNA, 16 bp, Artificial
  Synthetic
ttcrctcgccgttact

SEQ ID NO: 11 - DNA, 26 bp, Artificial
  Synthetic
atgtcaccacaaacagaaactaaagc

SEQ ID NO: 12 - DNA, 21 bp, Artificial
  Synthetic
catgtgcaatacgtgaatacc